(19) United States
(12) Patent Application Publication
(10) Pub. No.: US 2003/0139386 A1
(43) Pub. Date: Jul. 24, 2003

(54) Pharmaceutical compositions based on azetidine derivatives

(76) Inventors: Sophie Cote, Antony (FR); Valerie Bobineau, Antony (FR); Maria-Teresa Peracchia, Parie (FR)

Correspondence Address: Finnegan, Henderson, Farabow,, Garrett & Dunner, L.L.P., 1300 I Street, N.W., Washington, DC, 20005-3315, US

(21) Appl. No.: 10/323,933

(22) Filed: Dec. 20, 2002

Related U.S. Application Data
(60) Provisional application No. 60/353,952, filed on Feb. 5, 2002.

(30) Foreign Application Priority Data
Dec. 21, 2001 (FR) 16638

(51) Int. Cl. A61K 31/397

(52) U.S. Cl. 514/210.01; 514/210.2

(57) ABSTRACT

A stable pharmaceutical composition comprising at least one azetidine derivative of formula

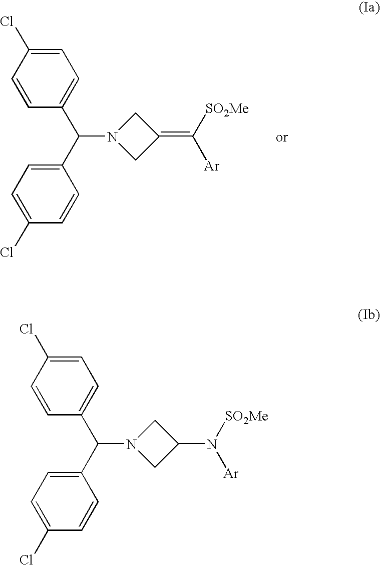

in which Ar is an aromatic or heteroaromatic group optionally substituted with one or more (C1-C4)alkyl, halogen, NO₂, CN, (C1-C4)alkoxy or OH groups, optionally in combination with one or more other active ingredients capable of potentiating the effects of the at least one azetidine derivative of formula (Ia) or (Ib), in a system comprising at most 2 principal excipients chosen from nonionic and hydrophilic surfactants capable of solubilizing the at least one azetidine derivative of formula (Ia) or (Ib) and, where appropriate, the one or more active ingredients potentiating the effects of the at least one azetidine derivative, and capable of causing the formation of a colloidal system, optionally supplemented with a second excipient of a lipophilic nature. The pharmaceutical compositions are advantageous because, for example, of the high affinity of the derivatives for cannabinoid receptors.

This application claims the benefit of priority under 35 U.S.C. § 119(e) to U.S. Provisional Application No. 60/353,952, filed on Feb. 5, 2002. This application also claims the benefit of priority under 35 U.S.C. § 119(a) to French Patent Application No. 01 16638, filed on Dec. 21, 2001.

The present invention relates to stable pharmaceutical compositions of azetidine derivatives.

The azetidine derivatives used in the pharmaceutical compositions according to the invention may be designated by the formula (Ia) or (Ib) below:

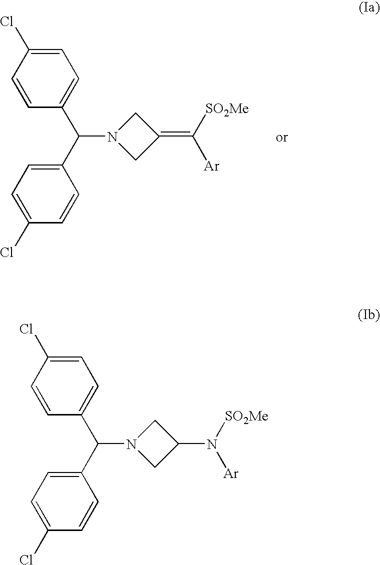

in which Ar is an aromatic or heteroaromatic group, wherein either group is unsubstituted or substituted with one or more groups chosen from (C1-C4)alkyl, halogen, NO₂, CN, (C1-C4)alkoxy and OH.

In the definition of the azetidine derivatives above, aromatic group is understood to mean a phenyl or naphthyl group, while heteroaromatic group is understood to mean a pyridyl, furyl, thienyl, thiazolyl, imidazolyl or oxazolyl group. Halogen is understood to mean fluorine, chlorine, bromine or iodine.

In international patent applications WO 00/15609, WO 01/64633, WO 01/64634 and WO 99/01451, there have been described azetidine derivatives of formula (Ia) or (Ib) and their applications. These azetidine derivatives are advantageous, for instance, for their high affinity for cannabinoid receptors and in particular CB1-type receptors.

Unfortunately, azetidine derivatives are products which are only very slightly water-soluble. Up until now, it was envisaged to administer the azetidine derivatives of formula (Ia) or (Ib), in particular by the oral route, in the form of tablets in formulations comprising, inter alia, cellulose, lactose and other excipients. However, such formulations are not always sufficiently well suited to these sparingly water-soluble products because of an excessively low bioavailability.

Numerous documents describe systems suitable for solubilizing and/or enhancing the bioavailability of hydrophobic active ingredients. However, the systems tested have so far proved ineffective for the preparation of pharmaceutical compositions containing azetidine derivatives defined above which are stable and bioavailable and in which the azetidine derivative is solubilized at an effective concentration.

J. Pharm Sciences, 89(8), 967 (2000) and Pharmaceutical Technology Europe, p. 20, September 2000, mention the formulation of active ingredients which are sparingly soluble in water, in medium-chain triglycerides. However, the trials carried out with formulations based on Miglyol® have given insufficient results from the point of view of their bioavailability.

Moreover, international application WO 95/24893 describes compositions comprising a digestible oil, a lipophilic surfactant and a hydrophilic surfactant which are intended for the formulation of hydrophobic active ingredients and for the enhancement of their bioavailability. Unfortunately, the above azetidine derivatives have proved too weakly bioavailable in this type of formulation. In particular, the formulation of such azetidine derivatives in a Miglyol®/Capryol®/Cremophor® system has also proved insufficient in vivo from the pharmacokinetic point of view.

It has now been found that it is possible to prepare chemically and physically stable pharmaceutical compositions comprising at least one derivative of formula (Ia) or (Ib), optionally in combination with one or more other active ingredients capable of potentiating the effects of the at least one azetidine derivative of formula (Ia) or (Ib), in a system comprising at most 2 principal excipients chosen from nonionic and hydrophilic surfactants capable of solubilizing the at least one azetidine derivative of formula (Ia) or (Ib) and, where appropriate, the active ingredient potentiating the effects of the at least one azetidine derivative, and of causing the formation of a colloidal system, and a second and lipophilic excipient. A principal excipient is understood to mean an excipient that solubilizes the at least one derivative of formula (Ia) or (Ib) at an effective concentration to render the pharmaceutical composition comprising the derivative or derivatives chemically and physically stable.

According to the invention, illustrative compositions comprise:

at least one active ingredient of formula (Ia) or (Ib),

optionally one or more other active ingredients capable of potentiating the effects of the at least one azetidine derivative of formula (Ia) or (Ib),

a nonionic and hydrophilic surfactant capable of solubilizing the at least one azetidine derivative of formula (Ia) or (Ib) and, where appropriate, the active ingredient potentiating the effects of the azetidine derivative, and capable of causing the formation of a colloidal system,

optionally a lipophilic surfactant having an HLB of less than 10, and

optionally additives chosen from stabilizing agents, preservatives, agents which make it possible to adjust the viscosity, and agents which can modify, for example, the organoleptic properties of the compositions.

According to the invention, the nonionic and hydrophilic surfactant capable of solubilizing the at least one azetidine derivative of formula (Ia) or (Ib) and, where appropriate, the active ingredient potentiating the effects of the azetidine derivative, and capable of causing the formation of a colloidal system, may be chosen from, for instance, solid or semisolid agents, which melt at low temperature (T° C.<60° C.), or from liquid agents, whose HLB ranges from 10 to 20, such as glycerides of polyethylene glycol and saturated fatty acids.

It is understood that, in the above definition, the saturated fatty acids may contain from 6 to 18 carbon atoms, and that the glycerides of polyethylene glycol (PEG) and saturated fatty acids may be of natural or synthetic origin.

By way of example, the nonionic and hydrophilic surfactant may be chosen from agents such as Labrasol® [caprylcaproyl macrogol-8 glyceride] and the Gelucire® products: Gelucire 44/14, Gelucire 50/13, [lauroyl (or stearoyl, palmitoyl) macrogol-32 glyceride].

According to an embodiment of the invention, the composition may also comprise a lipophilic surface-active agent having an HLB of less than 10 as a second principal excipient. This agent may be chosen from agents capable of enhancing the solubilization of the azetidine derivative of formula (Ia) or (Ib) and, if necessary, of the associated active ingredient. According to the invention, this agent may be chosen from, for instance, glycerides of polyethylene glycol and fatty acids, including unsaturated fatty acids, from esters of polyethylene glycol and fatty acids and from esters of fatty acids and sorbitol. It being understood that the above fatty acids may contain from 6 to 18 carbon atoms.

By way of example, the agent may be chosen from oleic acid, from the Labrafil® products [oleoyl (or lineoyl) macrogol-8 glycerides], for example Labrafil M1944CS, Capryol® (polyethylene glycol monocaprylate) or Span 20® (sorbitol monolaurate). The present list being given without limitation.

Among the excipients cited above, Labrasol®, Gelucire® and the Labrafil®/Labrasol® pair are illustrative.

It has also been demonstrated (but not published by the filing date of the present application) that for certain treatments such as, for example, obesity, it may be advantageous to administer the azetidine derivatives of formula (Ia) or (Ib) at the same time as sibutramine which causes a synergistic effect in the reduction of food consumption.

Sibutramine and its effects have been described in the references below: WO 90/061110; D. H. RYAN et al., Obesity Research, 3 (4), 553 (1995); H. C. JACKSON et al., British Journal of Pharmacology, 121, 1758 (1997); G. FANGHANEL et al., Inter. J. Obes., 24 (2), 144 (2000); G. A. BRAY et al., Obes. Res., 7(2), 189 (1999).

Moreover, for other treatments such as schizophrenia or the treatment of neurological disorders such as Parkinson's disease, it may be advantageous to administer the azetidine derivatives of formula (Ia) or (Ib) at the same time as one or more agents which activate dopaminergic neurotransmission in the brain. These combinations make it possible to potentiate the effects of a dopaminergic monotherapy (levodopa, dopaminergic agonists, and inhibitors of enzymes), and make it possible to reduce side effects, such as dyskinesia.

Among the dopaminergic agonists, the following products are illustrative: bromocriptine (Novartis), cabergoline (Pharmacia Corp.), adrogolide (Abbott Laboratories), BAM-1110 (Maruko Seiyaku Co Ltd), Duodopa® (Neopharma), L-dopa, dopadose (Neopharma), CHF1512 (Chiesi), NeuroCell-PD (Diacrin Inc), PNU-95666 (Pharmacia & Upjohn) ropinirole (GlaxoSmithKline Beecham), pramipexole (Boehringer Ingelheim), rotigotine (Discovery Therapeutics, Lohmann Therapie System), spheramine (Titan Pharmaceuticals), TV1203 (Teva pharmaceutical) and uridine (Polifarma).

It is understood that the compositions comprising, in addition, an active ingredient other than the azetidine derivative of formula (Ia) or (Ib) and capable of potentiating the effects thereof may contain a product as defined in the paragraphs above and that such compositions fall within the scope of the present invention.

According to the invention, the active ingredient of formula (Ia) or (Ib) can represent, for example, from 0.01 to 70% by weight of the total composition. For instance, it can represent from 0.05 to 50% by weight and from 0.1 to 20% by weight of the total composition.

It is understood that the dosage may vary according to the degree or the nature of the condition to be treated. Thus, the quantity of active product in a composition according to the invention will be determined such that a suitable dosage can be prescribed. As a result, the quantity of azetidine derivative of formula (Ia) or (Ib) varies as a function of its solubility in the mixture and also as a function of the appropriate dosage for the treatment of patients.

In humans, the daily doses administered by the oral route can range, for example, from 0.1 to 100 mg of azetidine derivative of formula (Ia) or (Ib). It is understood that, to choose the most appropriate dosage, there should be taken into account the weight of the patient, his general state of health, his age and all factors which may influence the efficacy of the treatment. The compositions may be prepared such that a unit dose contains, for instance, from 0.1 to 50 mg of active product.

Among the azetidine derivatives of formula (Ia) or (Ib), the following products are illustrative:

1-[bis(4-chlorophenyl)methyl]-3-[(3,5-difluorophenyl)(methylsulfonyl)methylene]azetidine);

N-{1-[bis(4-chlorophenyl)methyl]azetidin-3-yl}-N-pyrid-3-ylmethylsulfonamide;

N-{1-[bis(4-chlorophenyl)methyl]azetidin-3-yl}-N-(3,5-difluorophenyl)methylsulfonamide;

N-{1-[bis-(4-chlorophenyl)methyl]-azetidin-3-yl}-N-(6-chloropyrid-2-yl)-methylsulfonamide; and

N-{1-[bis-(4-chlorophenyl)methyl]azetidin-3-yl}-N-quinol-6-yl-methylsulfonamide.

It is understood that the compositions according to the invention, containing these products, are illustrative.

In the alternative, where a second active ingredient is introduced, the compositions may comprise, for instance, 0.2 to 50 mg in the case where the associated product is sibutramine. However, this quantity may optionally be lower and may vary, for instance, from 0.2 to 10 mg.

In the case where the associated product is L-dopa, the compositions may comprise, for instance, from 100 to 300 mg of this second active ingredient, for instance 250 mg.

The nonionic and hydrophilic surfactant capable of causing the formation of a colloidal system, may represent, for instance, from 20 to 100% relative to the total weight of the excipients present in the composition, for instance from 40 to 100%, and also for instance from 60 to 100%.

Where appropriate, when the composition also contains a lipophilic principal excipient having an HLB of less than 10, the quantity of this agent with a low HLB may represent, for instance, from 0.1 to 60% relative to the total weight of the excipients present in the composition, and from 1 to 40%.

When the compositions further comprise certain additional additives, the latter may be, for instance, stabilizing agents, preservatives, agents which make it possible to adjust the viscosity, and agents which can modify, for example, the organoleptic properties of the compositions.

The stabilizing agents may be, for example, antioxidants chosen from α-tocopherol, ascorbyl palmitate, BHT (butyl hydroxytoluene), BHA (butyl hydroxyanisole), propyl gallate and malic acid.

The preservatives may, by way of example, be chosen from sodium metabisulfite, propylene glycol, ethanol and glycerin.

Among the agents capable of adjusting the viscosity, there may be mentioned, for example, lecithins, phospholipids, propylene glycol alginate, sodium alginate and glycerin.

Among the agents capable of modifying the organoleptic properties of the composition are, by way of example, malic acid, fumaric acid, glycerin, vanillin and menthol.

When such additives are used, the latter may constitute, for instance, from 0.001% to 5% by weight of the total composition.

According to an embodiment of the invention, the pharmaceutical composition may be obtained by mixing, where appropriate, the principal excipients (after heating if necessary, in the case of solid or semisolid excipients), and then, if necessary, mixing with the additional additives, followed by the addition of the at least one azetidine derivative of formula (Ia) or (Ib) and, where appropriate, of the active ingredient capable of potentiating the effects of the at least one azetidine derivative of formula (Ia) or (Ib), and maintaining stirred in order to obtain a homogeneous mixture.

The use of this process is described in greater detail below in the examples.

The compositions according to the invention may be provided, for instance, in the liquid, solid or semipasty state.

The compositions according to the invention are suitable, for instance, for presentation in the form of hard gelatin capsules or soft gelatin capsules, or in the form of an oral solution.

The compositions according to the invention are advantageous, for instance, because of their good stability, both physically and chemically, and the enhancement of the bioavailablity which they offer upon oral administration of the at least one azetidine derivative of formula (Ia) or (Ib).

Additionally illustrative of the present invention are the compositions comprising:

at least one active ingredient of formula (Ia) or (Ib),

a nonionic and hydrophilic surfactant capable of solubilizing the at least one azetidine derivative of formula (Ia) or (Ib), and capable of causing the formation of a colloidal system,

optionally a lipophilic surfactant having an HLB of less than 10,

optionally additives chosen from stabilizing agents, preservatives, agents which make it possible to adjust the viscosity, and agents which can modify, for example, the organoleptic properties of the compositions.

According to another alternative of the invention, the illustrative compositions as defined above, which contain at least one active ingredient of formula (Ia) or (Ib), may be administered before, simultaneously with or after the administration of an active ingredient capable of potentiating the effects of the at least one azetidine derivative of formula (Ia) or (Ib).

It is understood that presentation kits comprising, on the one hand, an illustrative composition according to the invention as defined above and, on the other hand, a composition comprising the active ingredient capable of potentiating the effects of the at least one azetidine derivative of formula (Ia) or (Ib), also fall within the scope of the present invention. It is also understood that the presentation kits may contain, as compositions capable of potentiating the effects of the at least one azetidine derivative of formula (Ia) or (Ib), compositions comprising sibutramine, or comprising an agent which activates dopaminergic neurotransmission in the brain.

The following examples, given without limitation, illustrate compositions according to the present invention.

### EXAMPLE 1

The Labrasol/Labrafil M1944CS mixture, 60/40 (m/m) ratio, was prepared at room temperature (20° C.), by magnetic stirring for 15 minutes of 14.4 g of Labrasol and 9.6 g of Labrafil M1944CS in a beaker. A very good miscibility was observed. 200 mg of 1-[bis(4-chlorophenyl)methyl]-3-[(3,5-difluorophenyl)(methylsulfonyl)methylene]azetidine were introduced into another beaker, and adjusted to 20 g with the Labrasol/Labrafil M1944CS 60/40 mixture in order to obtain a final concentration of 10 mg/g of 1-[bis(4-chlorophenyl)methyl]-3-[(3,5-difluorophenyl)(methylsulfonyl)methylene]azetidine. The mixture of the 3 constituents was kept mechanically stirred (300 rpm) at room temperature for 2 hours in order to obtain complete dissolution of the 1-[bis(4-chlorophenyl)methyl]-3-[(3,5-difluorophenyl)(methylsulfonyl)methylene]azetidine. The solution obtained was distributed in 1 g fractions into sealed glass vials and stored at 5° C.

A satisfactory chemical and physical stability was demonstrated for 1 month at 5° C.

An enhancement of the pharmacokinetic parameters by a factor of at least 2.5 was observed in comparison with a composition of 1-[bis(4-chlorophenyl)methyl]-3-[(3,5-difluorophenyl)(methylsulfonyl)methylene]azetidine in Miglyol 812®.

### EXAMPLE 2

By carrying out the procedure as above in example 1, but starting with 16.8 g of Labrasol and 7.2 g of Labrafil M1944CS in order to manufacture the Labrasol/Labrafil M1944CS mixture at the 70/30 (m/m) ratio, a composition was prepared containing 200 mg of 1-[bis(4-chlorophenyl)methyl]-3-[(3,5-difluorophenyl)(methylsulfonyl)methylene]azetidine adjusted to 20 g with the Labrasol/Labrafil M1944CS 70/30 mixture, in order to obtain a final concentration of 10 mg/g of 1-[bis(4-chlorophenyl)methyl]-3-[(3,5-difluorophenyl)(methylsulfonyl)methylene]azetidine.

A satisfactory chemical and physical stability was demonstrated for 1 month at 5° C.

This composition was tested in an in vitro model, in comparison with the composition of example 1. 400 mg of the composition were incubated in 20 ml of medium simulating gastric fluid (reference USP). After an incubation of 2 hours at 37° C., an HPLC assay was carried out after filtration on a 2 μm filter, in order to determine the colloidal stability of the formulations.

The behavior of this composition was equivalent to the behavior of the composition of example 1.

### EXAMPLE 3

By carrying out the procedure as above in example 1, but starting with 19.2 g of Labrasol and 4.8 g of Labrafil M1944CS in order to manufacture the Labrasol/Labrafil M1944CS mixture at the 80/20 (m/m) ratio, a composition was prepared containing 200 mg of 1-[bis(4-chlorophenyl)methyl]-3-[(3,5-difluorophenyl)(methylsulfonyl)methylene]azetidine adjusted to 20 g with the Labrasol/Labrafil M1944CS 80/20 mixture, in order to obtain a final concentration of 10 mg/g of 1-[bis(4-chlorophenyl)methyl]-3-[(3,5-difluorophenyl)(methylsulfonyl)methylene]azetidine.

A satisfactory chemical and physical stability was demonstrated for 1 month at 5° C.

This composition was tested in an in vitro model, in comparison with the composition of example 1. 400 mg of the composition were incubated in 20 ml of medium simulating gastric fluid (reference USP). After an incubation of 2 hours at 37° C., an HPLC assay was carried out after filtration on a 2 μm filter, in order to determine the colloidal stability of the formulations.

The behavior of this composition was equivalent to the behavior of the composition of example 1.

### EXAMPLE 4

By carrying out the procedure as above in example 1, but starting with 21.6 g of Labrasol and 2.4 g of Labrafil M1944CS in order to manufacture the Labrasol/Labrafil M1944CS mixture at the 90/10 (m/m) ratio, a composition was prepared containing 200 mg of 1-[bis(4-chlorophenyl)methyl]-3-[(3,5-difluorophenyl)(methylsulfonyl)methylene]azetidine adjusted to 20 g with the Labrasol/Labrafil M1944CS 90/10 mixture, in order to obtain a final concentration of 10 mg/g of 1-[bis(4-chlorophenyl)methyl]-3-[(3,5-difluorophenyl)(methylsulfonyl)methylene]azetidine.

A satisfactory chemical and physical stability was demonstrated for 1 month at 5° C.

This composition was tested in an in vitro model, in comparison with the composition of example 1. 400 mg of the composition were incubated in 20 ml of medium simulating gastric fluid (reference USP). After an incubation of 2 hours at 37° C., an HPLC assay was carried out after filtration on a 2 μm filter, in order to determine the colloidal stability of the formulations.

The behavior of this composition was equivalent to the behavior of the composition of example 1.

### EXAMPLE 5

By carrying out the procedure as above in example 1, but starting with 24 g of Labrasol only, a composition was prepared containing 200 mg of 1-[bis(4-chlorophenyl)methyl]-3-[(3,5-difluorophenyl)(methylsulfonyl)methylene]azetidine adjusted to 20 g with Labrasol, in order to obtain a final concentration of 10 mg/g of 1-[bis(4-chlorophenyl)methyl]-3-[(3,5-difluorophenyl)(methylsulfonyl)methylene]azetidine.

A satisfactory chemical and physical stability was demonstrated for 1 month at 5° C.

This composition was tested in an in vitro model, in comparison with the composition of example 1. 400 mg of the composition were incubated in 20 ml of medium simulating gastric fluid (reference USP). After an incubation of 2 hours at 37° C., an HPLC assay was carried out after filtration on a 2 μm filter, in order to determine the colloidal stability of the formulations.

The behavior of this composition was equivalent to the behavior of the composition of example 1.

### EXAMPLE 6

By carrying out the procedure as above in example 1, but starting with 24 g of Gelucire 44/14 as a replacement for the Labrasol/Labrafil M1944CS mixture. Gelucire 44/14 was molten beforehand in the region of 55° C. 200 mg of 1-[bis(4-chlorophenyl)methyl]-3-[(3,5-difluorophenyl)(methylsulfonyl)methylene]azetidine were introduced into a beaker, and adjusted to 20 g with Gelucire 44/14, in order to obtain a final concentration of 10 mg/g of 1-[bis(4-chlorophenyl)methyl]-3-[(3,5-difluorophenyl)(methylsulfonyl)methylene]azetidine. The mixture of the 2 constituents was kept magnetically stirred (300 rpm) at 50-55° C. for 1 hour in order to obtain complete dissolution of 1-[bis(4-chlorophenyl)methyl]-3-[(3,5-difluorophenyl)(methylsulfonyl)methylene]azetidine. The mass was distributed into hard gelatin capsules which were stored overnight in a freezer at −20° C. The envelope of the hard gelatin capsules was then separated from the solid mass contained inside using a cutter. The samples were stored in sealed glass vials at 5° C.

A satisfactory chemical and physical stability was demonstrated for 1 month at 5° C.

This composition was tested in an in vitro model, in comparison with the composition of example 1. 400 mg of the composition were incubated in 20 ml of medium simulating gastric fluid (reference USP). After an incubation of 2 hours at 37° C., an HPLC assay was carried out after filtration on a 2 μm filter, in order to determine the colloidal stability of the formulations.

The behavior of this composition was equivalent to the behavior of the composition of example 1.

### EXAMPLE 7

A Labrasol/Labrafil M1944CS mixture, 60/40 (m/m) ratio, was prepared at room temperature (20° C.), by magnetic stirring for 15 minutes of 30 g of Labrasol and 20 g of Labrafil M1944CS in a beaker. A very good miscibility was observed. 20 mg of 1-[bis(4-chlorophenyl)methyl]-3-[(3,5-difluorophenyl)(methylsulfonyl)methylene]azetidine were introduced into a graduated flask of 10 ml. After having adjusted to 10 ml with the necessary quantity of Labrasol/Labrafil M1944CS 60/40 mixture, the mixture of the 3 constituents was kept magnetically stirred (500 rpm) at room temperature for 2 hours in order to obtain complete dissolution of the 1-[bis(4-chlorophenyl)methyl]-3-[(3,5-difluorophenyl)(methylsulfonyl)methylene]azetidine. The solution obtained was distributed in 2.5 ml fractions into sealed glass vials and stored at 5° C.

This formulation, at the concentration of 2 mg/ml of 1-[bis(4-chlorophenyl)methyl]-3-[(3,5-difluorophenyl)(methylsulfonyl)methylene]azetidine, was used to carry out pharmacokinetic studies in monkeys after oral administration at a dose of 1 mg/kg. To do this, this solution was diluted to one tenth in apple juice in order to facilitate administration to the animal. The emulsion obtained after dilution was physically and chemically stable for at least one hour.

### EXAMPLE 8

A Labrasol/Labrafil M1944CS mixture, 60/40 (m/m) ratio, was prepared at room temperature (20° C.), by magnetic stirring for 15 minutes of 30 g of Labrasol and 20 g of Labrafil M1944CS in a beaker. A very good miscibility was observed. 20 mg of N-{1-[bis(4-chlorophenyl)methyl]azetidin-3-yl}-N-(3,5-difluorophenyl)methylsulfonamide are introduced into a graduated flask of 10 ml. After having adjusted to 10 ml with the necessary quantity of Labrasol/Labrafil M1944CS 60/40 mixture, the mixture of the 3 constituents was kept magnetically stirred (500 rpm) at room temperature for 2 hours in order to obtain complete dissolution of the N-{1-[bis(4-chlorophenyl)methyl]azetidin-3-yl}-N-(3,5-difluorophenyl)methylsulfonamide. The solution obtained was distributed in 2.5 ml fractions into sealed glass vials and stored at 5° C.

This formulation, at the concentration of 2 mg/ml of N-{1-[bis(4-chlorophenyl)methyl]azetidin-3-yl}-N-(3,5-difluorophenyl)methylsulfonamide, was used to carry out pharmacokinetic studies in monkeys after oral administration at a dose of 1 mg/kg. To do this, this solution was diluted one tenth in apple juice in order to facilitate administration to the animal. The emulsion obtained after dilution was physically and chemically stable for at least one hour.

### EXAMPLE 9

A Labrasol/Labrafil M1944CS mixture, 60/40 (m/m) ratio, was prepared at room temperature (20° C.), by magnetic stirring for 15 minutes of 30 g of Labrasol and 20 g of Labrafil M1944CS in a beaker. A very good miscibility was observed. 10 mg of N-{1-[bis(4-chlorophenyl)methyl]azetidin-3-yl}-N-pyrid-3-ylmethyl-sulfonamide was introduced into a graduated flask of 10 ml. After having adjusted to 10 ml with the necessary quantity of Labrasol/Labrafil M1944CS 60/40 mixture, the mixture of the 3 constituents was kept magnetically stirred (500 rpm) at room temperature for 2 hours in order to obtain complete dissolution of the N-{1-[bis-(4-chlorophenyl)methyl]azetidin-3-yl}-N-pyrid-3-ylmethylsulfonamide. The solution obtained was distributed in 2.5 ml fractions into sealed glass vials and stored at 5° C.

This formulation, at the concentration of 1 mg/ml of N-{1-[bis(4-chlorophenyl)methyl]azetidin-3-yl}-N-pyrid-3-ylmethylsulfonamide was used to carry out pharmacological studies in rats after oral administration at a dose of 1 mg/kg.

### EXAMPLE 10

By carrying out the procedure as above in example 9, but starting with 30 mg of N-{1-[bis(4-chlorophenyl)methyl]azetidin-3-yl}-N-pyrid-3-ylmethyl-sulfonamide adjusted to 10 ml with the Labrasol/Labrafil M1944CS 60/40 mixture, a solution was prepared containing 3 mg/ml of N-{1-[bis(4-chlorophenyl)methyl]azetidin-3-yl}-N-pyrid-3-ylmethyl-sulfonamide.

This formulation at the concentration of 3 mg/ml of N-{1-[bis(4-chlorophenyl)methyl]azetidin-3-yl}-N-pyrid-3-ylmethylsulfonamide was used to carry out pharmacological studies in rats after oral administration at a dose of 3 mg/kg.

### EXAMPLE 11

By carrying out the procedure as above in example 9, but starting with 50 mg of N-{1-[bis(4-chlorophenyl)methyl]azetidin-3-yl}-N-pyrid-3-ylmethyl-sulfonamide adjusted to 5 ml with the Labrasol/Labrafil M1944CS 60/40 mixture, a solution was prepared containing 10 mg/ml of N-{1-[bis(4-chlorophenyl)methyl]azetidin-3-yl}-N-pyrid-3-ylmethylsulfonamide.

This formulation at the concentration of 10 mg/ml of N-{1-[bis(4-chlorophenyl)methyl]azetidin-3-yl}-N-pyrid-3-ylmethylsulfonamide was used to carry out pharmacological studies in rats after oral administration at a dose of 10 mg/kg.

We claim:

1. A stable pharmaceutical composition comprising
at least one azetidine derivative of formula: 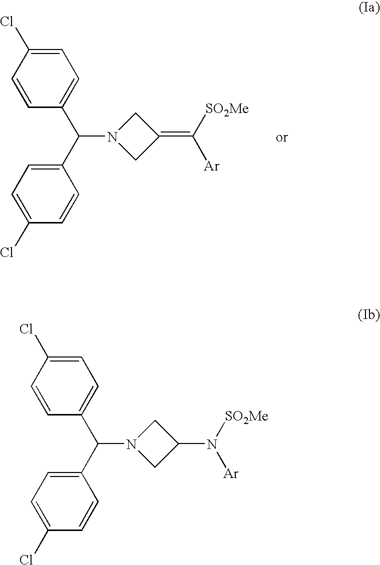
in which Ar is an aromatic or heteroaromatic group, wherein the aromatic or heteroaromatic group is unsubstituted or substituted with one or more groups chosen from (C1-C4)alkyl, halogen, NO₂, CN, (C1-C4)alkoxy and OH, and
1 principal excipient, wherein the 1 principal excipient is a nonionic and hydrophilic surfactant capable of solubilizing the at least one azetidine derivative of formula (Ia) or (Ib), and capable of causing the formation of a colloidal system.

2. A stable pharmaceutical composition comprising
at least one azetidine derivative of formula: 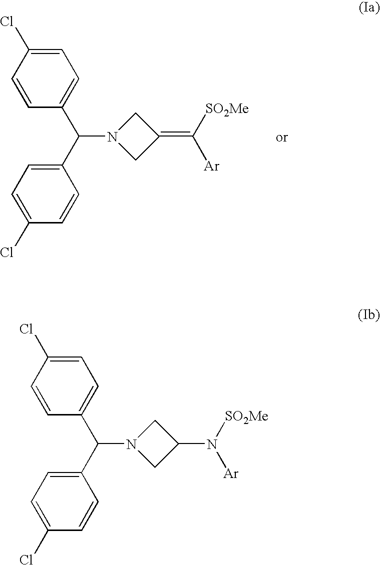
in which Ar is an aromatic or heteroaromatic group, wherein the aromatic or heteroaromatic group is unsubstituted or substituted with one or more groups chosen from (C1-C4)alkyl, halogen, NO₂, CN, (C1-C4)alkoxy and OH, and
2 principal excipients, wherein
the first principal excipient is a nonionic and hydrophilic surfactant capable of solubilizing the at least one azetidine derivative of formula (Ia) or (Ib), and capable of causing the formation of a colloidal system, and
the second principal excipient is a lipophilic excipient.

3. The stable pharmaceutical composition as claimed in claim 1, which further comprises one or more other active ingredients capable of potentiating the effects of the at least one azetidine derivative of formula (Ia) or (Ib), and wherein the principal excipient is capable of solubilizing the one or more other active ingredients.

4. The stable pharmaceutical composition as claimed in claim 2, which further comprises one or more other active ingredients capable of potentiating the effects of the at least one azetidine derivative of formula (Ia) or (Ib), and wherein the principal excipients are capable of solubilizing the one or more other active ingredients.

5. The stable pharmaceutical composition as claimed in claim 1, which further comprises one or more additives chosen from stabilizing agents, preservatives, agents which make it possible to adjust the viscosity of the composition, and agents which can modify the organoleptic properties of the composition.

6. The stable pharmaceutical composition as claimed in claim 2, which further comprises one or more additives chosen from stabilizing agents, preservatives, agents which make it possible to adjust the viscosity of the composition, and agents which can modify the organoleptic properties of the composition.

7. The stable pharmaceutical composition as claimed in claim 2, wherein the second and lipophilic principal excipient has an HLB of less than 10.

8. The stable pharmaceutical composition as claimed in claim 5, which further comprises one or more other active ingredients capable of potentiating the effects of the azetidine derivative of formula (Ia) or (Ib), and wherein the principal excipient is capable of solubilizing the one or more other active ingredients.

9. The stable pharmaceutical composition as claimed in claim 6, which further comprises one or more other active ingredients capable of potentiating the effects of the azetidine derivative of formula (Ia) or (Ib), and wherein the principal excipients are capable of solubilizing the one or more other active ingredients.

10. The stable pharmaceutical composition as claimed in claim 1, wherein the aromatic group of the at least one azetidine derivative of formula (Ia) or (Ib) is an unsubstituted phenyl or naphthyl group.

11. The stable pharmaceutical composition as claimed in claim 2, wherein the aromatic group of the at least one azetidine derivative of formula (Ia) or (Ib) is an unsubstituted phenyl or naphthyl group.

12. The stable pharmaceutical composition as claimed in claim 1, wherein the heteroaromatic group of the at least one azetidine derivative of formula (Ia) or (Ib) is an unsubstituted pyridyl, furyl, thienyl, thiazolyl, imidazolyl or oxazolyl group.

13. The stable pharmaceutical composition as claimed in claim 2, wherein the heteroaromatic group of the at least one azetidine derivative of formula (Ia) or (Ib) is an unsubstituted pyridyl, furyl, thienyl, thiazolyl, imidazolyl or oxazolyl group.

14. The stable pharmaceutical composition as claimed in claim 1, wherein the nonionic and hydrophilic surfactant is chosen from glycerides of polyethylene glycol and saturated fatty acids, whose HLB ranges from 10 to 20.

15. The stable pharmaceutical composition as claimed in claim 2, wherein the nonionic and hydrophilic surfactant is chosen from glycerides of polyethylene glycol and saturated fatty acids, whose HLB ranges from 10 to 20.

16. The stable pharmaceutical composition as claimed in claim 14, wherein the glycerides of polyethylene glycol and saturated fatty acids are glycerides of polyethylene glycol and saturated fatty acids containing from 6 to 18 carbon atoms.

17. The stable pharmaceutical composition as claimed in claim 15, wherein the glycerides of polyethylene glycol and saturated fatty acids are glycerides of polyethylene glycol and saturated fatty acids containing from 6 to 18 carbon atoms.

18. The stable pharmaceutical composition as claimed in claim 14, wherein the glycerides are of natural origin.

19. The stable pharmaceutical composition as claimed in claim 15, wherein the glycerides are of natural origin.

20. The stable pharmaceutical composition as claimed in claim 14, wherein the glycerides are of synthetic origin.

21. The stable pharmaceutical composition as claimed in claim 15, wherein the glycerides are of synthetic origin.

22. The stable pharmaceutical composition as claimed in claim 2, wherein the second and lipophilic principal excipient is chosen from glycerides of polyethylene glycol and unsaturated fatty acids, from esters of polyethylene glycol and fatty acids and from esters of fatty acids and sorbitol, having an HLB of less than 10.

23. The stable pharmaceutical composition as claimed in claim 1, wherein the principal excipient consists of a) caprylcaproyl macrogol-8 glyceride, or b) lauroyl, stearoyl, or palmitoyl macrogol-32 glyceride.

24. The stable pharmaceutical composition as claimed in claim 2, wherein the principal excipients consist of oleoyl or lineoyl macrogol-8 glyceride paired with caprylcaproyl macrogol-8 glyceride.

25. The stable pharmaceutical composition as claimed in claim 1, wherein the at least one azetidine derivative is present in an amount ranging from 0.01 to 70% by weight of the total composition.

26. The stable pharmaceutical composition as claimed in claim 2, wherein the at least one azetidine derivative is present in an amount ranging from 0.01 to 70% by weight of the total composition.

27. The stable pharmaceutical composition as claimed in claim 2, wherein the nonionic and hydrophilic surfactant is present in an amount of at least 20% relative to the total weight of the excipients in the composition.

28. The stable pharmaceutical composition as claimed in claim 2, wherein the second and lipophilic principal excipient is present in an amount ranging from 0.1 to 60% relative to the total weight of the excipients in the composition.

29. A process for preparing a stable pharmaceutical composition as claimed in claim 1, which comprises
preparing the principal excipient with any additional additives, wherein the principal excipient is heated in the case of the excipient being in solid or semisolid form,
adding the at least one azetidine derivative of formula (Ia) or (Ib) and, optionally, one or more additional active ingredients capable of potentiating the effects of the at least one azetidine derivative of formula (Ia) or (Ib),and
stirring the combined mixture in order to obtain a homogeneous mixture.

30. A process for preparing a stable pharmaceutical composition as claimed in claim 2, which comprises
preparing a mixture of the 2 principal excipients with any additional additives, wherein one or both of the principal excipients are heated in the case of the excipient or excipients being in solid or semisolid form,
adding the at least one azetidine derivative of formula (Ia) or (Ib) and, optionally, one or more additional active ingredients capable of potentiating the effects of the at least one azetidine derivative of formula (Ia) or (Ib),and
stirring the combined mixture in order to obtain a homogeneous mixture.

31. A presentation kit comprising a stable pharmaceutical composition as claimed in claim 1 and a composition comprising one or more active ingredients capable of potentiating the effects of the at least one azetidine derivative of formula (Ia) or (Ib).

32. A presentation kit comprising a stable pharmaceutical composition as claimed in claim 2 and a composition comprising one or more active ingredients capable of potentiating the effects of the at least one azetidine derivative of formula (Ia) or (Ib).

33. The presentation kit as claimed in claim 31, wherein the composition comprising one or more active ingredients capable of potentiating the effects of the at least one azetidine derivative of formula (Ia) or (Ib) is a composition comprising sibutramine.

34. The presentation kit as claimed in claim 32, wherein the composition comprising one or more active ingredients capable of potentiating the effects of the at least one azetidine derivative of formula (Ia) or (Ib) is a composition comprising sibutramine.

35. The presentation kit as claimed in claim 31, wherein the composition comprising one or more active ingredients capable of potentiating the effects of the at least one azetidine derivative of formula (Ia) or (Ib) is a composition comprising an agent that activates dopaminergic neurotransmission in the brain.

36. The presentation kit as claimed in claim 32, wherein the composition comprising one or more active ingredients capable of potentiating the effects of the at least one azetidine derivative of formula (Ia) or (Ib) is a composition comprising an agent that activates dopaminergic neurotransmission in the brain.
